(19) United States
(12) Patent Application Publication
(10) Pub. No.: US 2017/0231612 A1
(43) Pub. Date: Aug. 17, 2017

(54) FEMORAL ELEVATOR DEVICE

(71) Applicant: Zafer Termanini, Port Saint Lucie, FL (US)

(72) Inventor: Zafer Termanini, Port Saint Lucie, FL (US)

(21) Appl. No.: 15/043,752

(22) Filed: Feb. 15, 2016

(51) Int. Cl. A61B 17/02

(52) U.S. Cl. CPC A61B 17/025; A61B 2017/0275

(57) ABSTRACT

A proximal femoral bone elevating device used during a total hip replacement surgical procedure for primarily lifting the proximal femoral bone outside the surgical wound to allow the surgeon for adequately ream and prepare the lifted bone for insertion of trials and implant into the medullary cavity of the proximal femur. The device comprises a main gearbox, a vertical shaft and a bone hook attachment which is placed under the trochanteric region of the proximal femur. The main gearbox is firmly attached over the sterile drapes to the side rails of conventional operating tables. Turning the crank handle will raise the vertical shaft and the bone hook thereby lifting the proximal femur outside the surgical wound.

### FIELD OF THE INVENTION

The disclosure relates generally to a medical device used during hip replacement surgery, and more particularly as a device for elevating the proximal portion of the femur during minimally invasive hip replacement surgical procedures.

### BACKGROUND OF THE INVENTION

Total hip replacement has been one of the most commonly performed surgical orthopedic procedures and has been regarded as among the most valued advances in the history of orthopedics. It has been remarkably successful in replacing hip joints destroyed by degenerative disease, trauma and rheumatoid arthritis. Recently there have been significant advancements in the surgical techniques allowing the performance of implant insertion through minimally invasive smaller skin incisions using the anterior approach. In order to approach the hip joint anteriorly, the operating surgeon must use specialized instruments and retractors which will be inserted through much smaller incision anteriorly as compared to much larger incisions of conventional lateral or posterior approaches. This is technically challenging and more demanding since the operative field is small and compared to a deep hole. Several specialized retractors are commonly used to provide retraction and distraction of the muscles and tissue. Once the femoral head and neck are osteotomised and removed, the operating surgeon will then ream and clean the femoral intramedullary canal in preparation for the insertion of the femoral implant. Reamers and broaches are used to clean the medullary canal. During said reaming, the surrounding muscles and soft tissues are subjected to significant undue trauma and injury. Often, the operated leg is placed in extension and external rotation by lowering the foot portion of the operative table. This simply does not bring the proximal portion of the femur cut side the wound to allow the surgeon the ream the medullary canal without causing undue damage and attrition to the adjacent muscles and soft tissue.

When hand held retractors are used to raise the proximal femur to facilitate reaming of the medullary canal, they will be levered against the edge of the wound causing significant damage and maceration of the skin and subcutaneous tissue. They will apply undue pressure onto the surrounding muscles causing unrepairable damages. Other lifting means use electrical motor in a form of a winch to jack up the proximal femur in order to expose its proximal end through the wound. Said means are expensive and attached to the table as part of the frame. There is usually one motor attached to the right side of the table and one to the left side of the table. This system poses sterility concerns since the moving post has to penetrate through the drapes.

Another form of mechanical femoral elevator is available in the industry designed by Richard Wixson, where lifting of the femur is accomplished by an overhead unit attached to the table, comprising a vertical and horizontal rods as well as femoral hook attached to a T handle bolt. When said bolt is turned the hook will raise the proximal femur. However, said overhead elevator is bulky, cumbersome and prone for contamination during the surgical procedure due to its size and height as well as proximity to the overhead surgical light. Furthermore, said overhead elevator stands in the way of the surgeon increasing and may come too close to the non sterile overhead operating light thereby significantly increasing the chance of contamination. Other electromechanical device are solidly attached to the main frame of the specialized surgical table and remain non sterile. Their vertical shaft has to penetrate and violate the sterile draping representing a significant contamination concern. Furthermore. Since the device is permanently attached to the table, it cannot be used with any other conventional and commonly used surgical tables.

While these devices may be suitable for the particular purpose to which they address, they are not suitable for providing non-traumatic simplified method for elevating the proximal femur through minimal skin incision during total hip joint replacement.

A major improvement provided by the present invention is a simple mechanical jack and simplified femoral hook attachment. The device is easily attached over the sterile surgical drapes to the right or left sides of the surgical table and providing proximal femoral lift without any traumatic leverage against the edge of the wound and without any bulky and cumbersome overhead suspended device. Furthermore, the device will not interfere and stand in the way of the surgeon. Additionally, the hook is curved and shaped to contour the bone in a way that avoid any undue pressure and injury to the muscles and surrounding tissue.

### BRIEF DESCRIPTION OF THE INVENTION

During hip replacement surgery, the hip joint is routinely dislocated and the femoral head must be elevated and lifted out of the wound in order to prepare it for implantation. The head is transected and the proximal femur is raised and supported outside the wound using various hand held elevators and retractors, which often apply undue damage and injury to the surrounding soft tissues and muscles since they rely on surrounding soft tissue for leverage in order to support the femur. Other available elevators on the market are space occupying and too bulky. They provide traction from above to lift the proximal femur. However, they stand in the way and interfere with the ability of the surgeon to adequately access the wound. In view of the forgoing disadvantages inherent in conventional femoral lifting devices, the present invention provides a new mechanical jack, which provides strong lifting mechanism to the proximal portion of femoral bone allowing it to be raised and protrude through the surgical wound in order for the operating surgeon to ream and prepare the proximal femur for the insertion of the femoral stem. Said insertion would be very difficult if the proximal portion of the femur remains deeply imbedded in the surgical wound. Excessive pulling of the bone using retractors may result in fracture of the bone, which represents a considerable undesired complication.

The general purpose of the present invention, which will be described subsequently in greater detail, is to provide a new mechanical jack comprising a gearbox, which will be firmly attached over the sterile drapes to side rails of any conventional operating table. A vertical shaft passing through the gearbox and having a hook at its upper end. Said hook is passed under the proximal femur and lesser trochanteric region. The new system has many of the advantages of the conventional femoral elevating tools and many novel features that are not anticipated, rendered obvious, suggested or even implied by any of prior art conventional devices, either alone or in any combination thereof.

To attain this, the present invention generally comprises a main gearbox containing a plurality of gears. At least one of the gears making contact d engages with the gears of the vertical shaft passing through the main gearbox. Furthermore, a crank handle can be inserted into the small gear through a side opening. The vertical shaft having gear teeth that will engage with the large gear located in the main gearbox in order to provide the appropriate lifting mechanical advantage. The main gearbox can be slidingly attached via dovetail retention grooves to a fixation device attachment comprising two clamps. Said clamps can be firmly attached to the side rails available on all conventional operating tables. The proximal upper end of the vertical shaft provide a T shaped joint having an circular hollow channel allowing the horizontal shaft and femoral bone hook to be slidingly adjusted according to the patient's size. A rotating joint is interposed between the horizontal and vertical shaft to allow rotation freedom.

### BRIEF DESCRIPTION OF THE DRAWINGS

Various objects, features and advantages of the present invention will become fully appreciated as the same become better understood when considered in conjunction with the accompanying drawings, in which the same reference number is used throughout the several views to refer to an identical or similar, wherein:

FIG. 1 is perspective oblique view of a preferred embodiment of the femoral elevator device and its attachment made in accordance with the subject invention.

FIG. 2 is a side cross view of the gearbox showing the sliding vertical shaft gears engaged with the large circular gear of the gearbox.

FIG. 3 is a perspective side view of the fixation component showing the sliding dovetail and the clamps.

FIG. 4 is a perspective top view of the gearbox with fixation attachment.

### DETAILED DESCRIPTION OF THE INVENTION

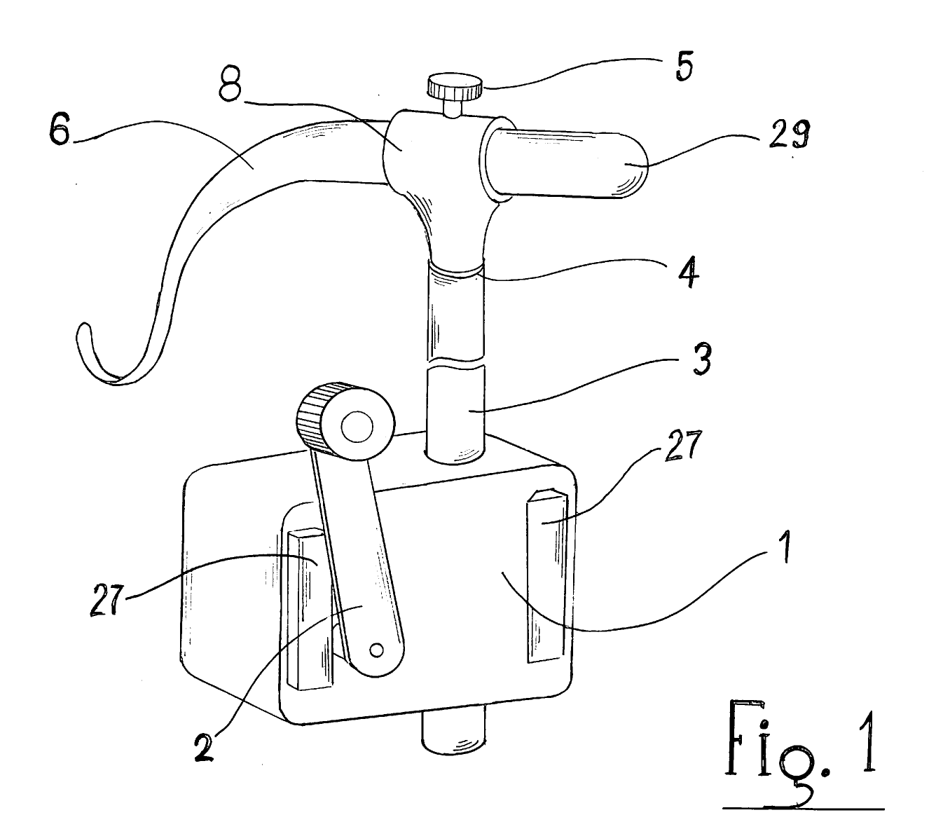

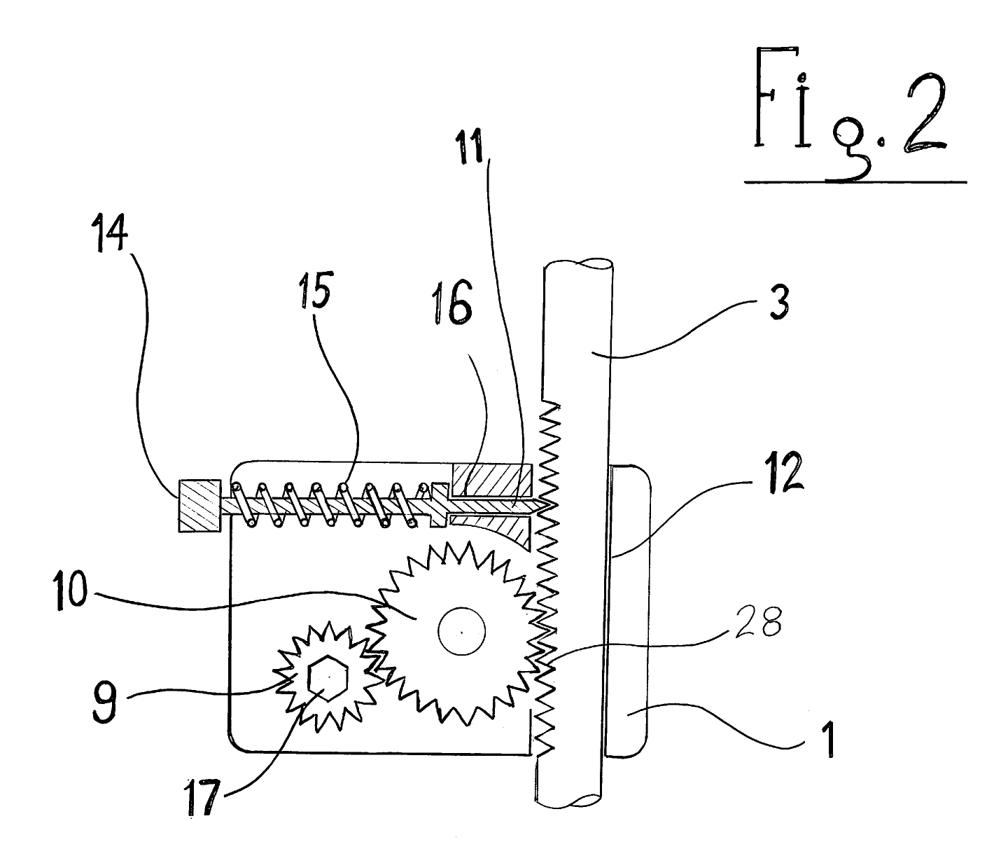

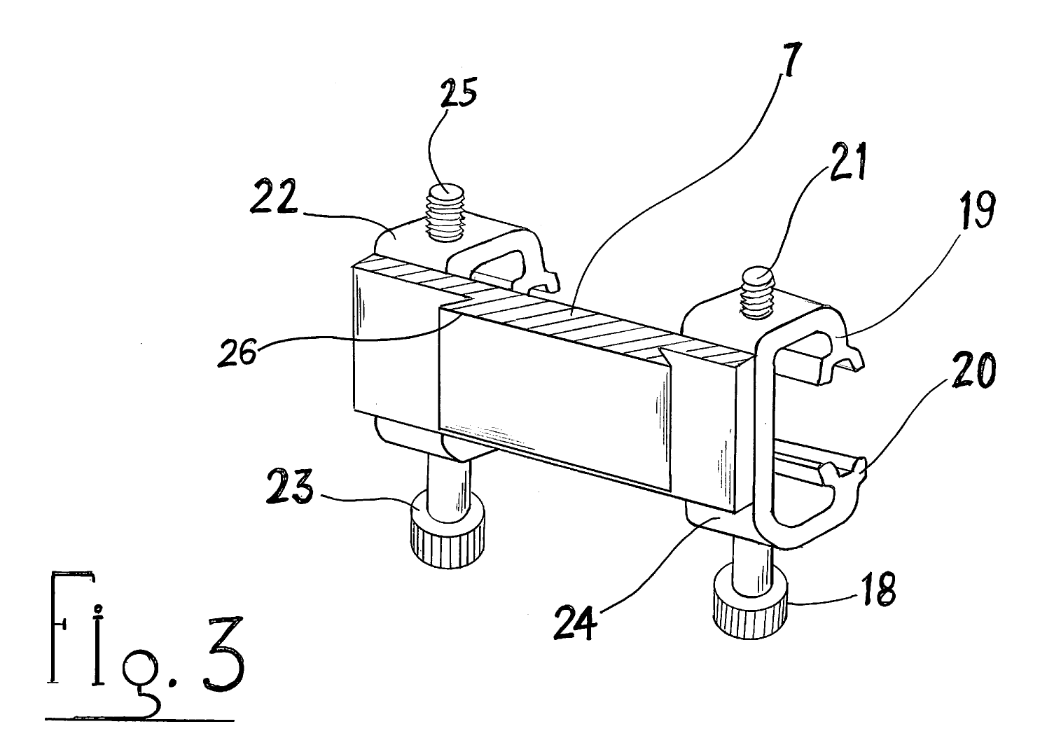

Turning now descriptively to the drawings, in which similar references characters denote similar elements throughout the several views, the attached figures illustrate a mechanical elevating femoral jack and attachment device suitable for elevating the proximal end of the femur outside the surgical wound in order to allow the operating surgeon to perform the necessary preparation of the femur for insertion of the trials prior to implanting the prosthesis. FIG. 1-4 illustrate a preferred embodiment of the femoral elevating jack. As seen best in FIG. 1, the device comprises a main gear box 1 and a vertical shaft 3 sliding through a vertical channel 12 in the main gearbox. Said vertical shaft can have a circular, square or hexagonal shape cross-section. A crank handle 2 having an hexagonal end can be inserted through the side of the main gear and into the hexagonal recess 17 of small gear 9. Turning the handle will rotate the small gear 9 as well as the large gear 10 providing a significant mechanical advantage. The teeth of large gear engage with the teeth of the vertical shaft 3 passing through the vertical channel 12. This mechanism allows the vertical shaft to be slidingly raised or lowered. Additionally, in order to prevent accidental movement of the vertical shaft, a locking mechanism is described in FIG. 2 where a tongue 11 contained in channel 16 of the main gearbox will protrude in the vertical channel 12 and interlock with the gear teeth 28 of the vertical shaft 3. This will prevent any accidental or unwanted movement of the vertical shaft. Spring 15 will keep locking tongue 11 solidly engaged. Pulling the release knob 14 will release tongue 11 from the vertical shaft gears allowing the vertical shaft to move and slide up or down. Other mechanical locking mechanism known in the art may be used. In addition, the proximal upper end of the vertical shaft provide a shaped clamp 8 having an circular hollow channel allowing transverse bar 29 and femoral hook 6 to be slidingly adjusted according to the patient's size. Additionally, the T shaped clamp 8 is attached to the vertical shaft via a rotating joint 4. Tightening knob 5 secures the position of the horizontal shaft 29 and femoral hook 6, as seen in FIG. 1.

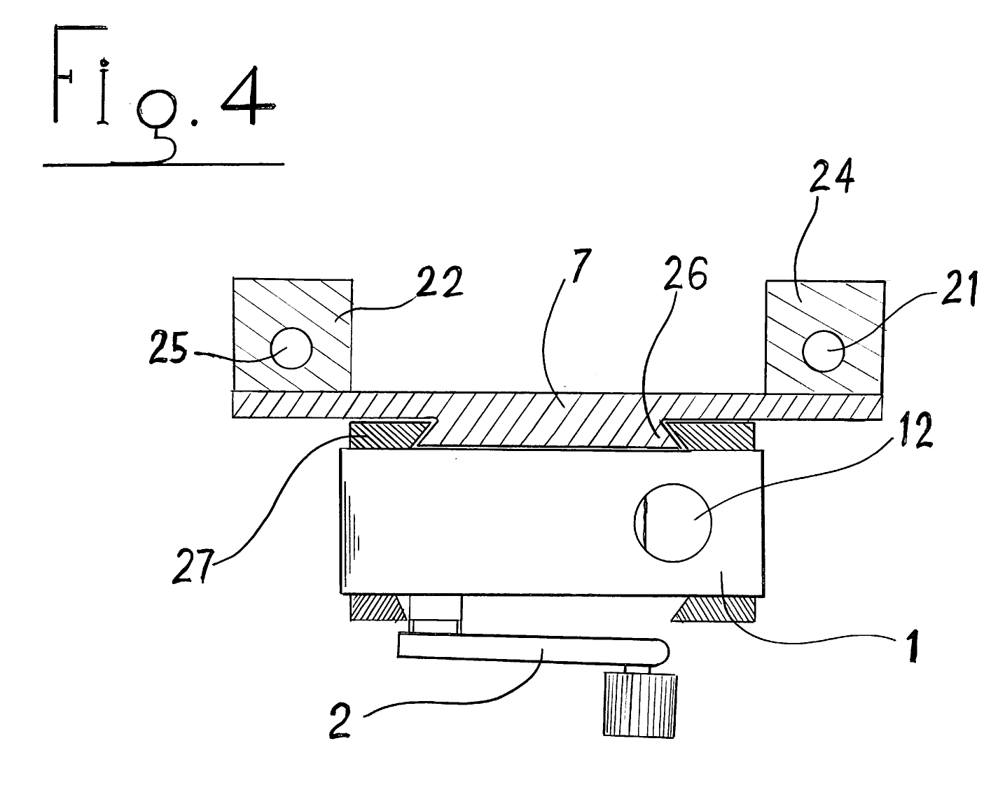

Furthermore, the main gearbox is firmly attached to the side rails of any conventional operating table using a fixation attachment as seen in FIG. 4. This is accomplished by having two vertically placed ridges 27 on each side of the main gearbox, forming a female dove tail recess configuration which will slidingly lock with male dove tail 26 of the attachment plate 7. The vertical female dovetail components are placed on both sides of the gear box to allow the use of the fixation attachment on the right and left side of the table. Said component firmly attaches to the side rails of conventional operating table using two C clamps 22 and 24 located on either end of the fixation plate 7. The described C clamps having upper 19 and lower 20 jaws that will fit over the sterile surgical drapes and conformably hug the top and the bottom edges of side rails of the surgical table. Said C clamps having screws 21 and 25 transfixing the C clamps and attached to knob 18 and 23. Once the C clamps are applied over the side rails, turning the knobs 18 and 23 will squeeze the Clamps onto the rail and solidly attach the fixation plate and the gearbox to the surgical table.

It is to be understood that the above description is intended to be illustrative and not restrictive. Although numerous characteristics and advantages of various embodiments as described herein have been set forth in the foregoing description, together with details of the structure and function of various embodiments, many other embodiments and changes to details will be apparent to those of skilled in the art upon reviewing the above description. For example, some or all of the gearbox and fixation attachment can he made out of ceramic, radio opaque plastic or metallic alloy. Other embodiment made out of recyclable plastic will allow the device to be disposable and intended for single use. Therefore, the scope of the invention should be, determined with reference to the appended claims, along with the full scope of equivalents to which such claims are entitled.

1. A femoral lifting device adapted to be removably mountable upon one or more side rails of a conventional operating table the femoral lifting device comprising:
a gearbox having a crank handle;
a geared slideable shaft passing vertically from within the gearbox;
a T shaped clamp mounted upon and attached to the geared slideable shaft via an intermediate rotating joint allowing for the T-shaped clamp to rotate with respect to the geared slideable shaft,
a femoral hook slideably affixed within the T-shaped clamp and rotatable about the geared slideable shaft, the femoral hook having a part adapted to be inserted into a surgical wound which is contoured to engage a proximal end of a femur of a patient; and,
a fixation attachment plate removably attached to the gearbox, the fixation attachment plate adapted to removably mount the gearbox to side rails of an operating table,
wherein, in operation, as actuation of the crank handle moves the T-shaped clamp in a direction away from the gearbox, a part of the T-shaped clamp engaging the femoral hook remains substantially perpendicular to a top surface of the operating table, and the part of the femoral hook contoured to engage a proximal end of a femur and which is in engagement therewith elevates and lifts a part of the femur outside the surgical wound without causing traumatic leverage against the end of the surgical wound.

2. The femoral lifting device of claim 1, wherein
the gearbox contains at least two or more gears with at least one of said gears engaging with gear teeth of the geared slideable shaft.

3. The femoral lifting device of claim 2, wherein
the crank handle has a hexagonal end configured to engage a corresponding hexagonal recess in the first gear, and the gearbox an opening on each of a pair of opposite surfaces which receive the crank handle.

4. The femoral lifting device of claim 1, wherein
the geared slideable shaft has a substantially circular, octagonal or square cross section.

5. The femoral lifting device of claim 1, wherein
the geared slideable shaft comprises gear teeth along a side thereof, the gear teeth configured to engage with corresponding teeth of the large a gear located in the gearbox.

6. The femoral lifting device of claim 1, wherein
the fixation attachment plate comprises a male dovetail on one side configured to slidably and firmly attach to a corresponding female dovetail recess on the gearbox.

7. The femoral lifting device of claim 1, wherein
at least one component of the device is made of one of ceramic, plastic or metallic alloy.

8. The orthopedic surgical femoral lifting device of claim 6, wherein
the fixation attachment plate comprises two squeezable C-clamps each having an upper and a lower jaw, each C-clamp located at a respective end of the fixation attachment plate and situated on a side opposite to the male dovetail, said C-clamps can firmly be attached by squeezing to the side rails of said operating table.

9. The femoral lifting device of claim 1, wherein
the femoral hook comprises a hook having a form of one of a semi-circle or having square angles.

10. The femoral lifting device of claim 8, wherein
each of the C-clamps has a knob and a rotatable shaft that can be actuated to cause a C-clamp to squeeze and attach to a side rail of an said operating table.

11. The femoral lifting device of claim 1, wherein the gearbox further comprises:
a locking mechanism which includes, a tongue located in a channel of the gearbox; and a spring urging the tongue to the geared slideable shaft between adjacent gear teeth.

12. The femoral lifting device of claim 1, wherein the geared slideable shaft has a rack of gear teeth.

13. The femoral lifting device of claim 1, wherein the geared slideable shaft passes vertically from the gearbox without rotation of the geared slideable shaft.

14. The femoral lifting device of claim 1, wherein the femoral hook is contoured to engage the trochanteric region of the femur.

15. The femoral lifting device of claim 11, wherein the tongue when engaged between adjacent gear teeth limits any linear motion of the geared slideable shaft passing vertically from within the gearbox.
